(19) United States
(12) Patent Application Publication
(10) Pub. No.: US 2012/0010475 A1
(43) Pub. Date: Jan. 12, 2012

(54) INTEGRATED DISPLAY AND CONTROL FOR MULTIPLE MODALITIES

(76) Inventors: Markus Rossmeier, Bamberg (DE); Dietrich Till, Forchheim (DE); Karl Lorenz, Erlangen (DE); Gerhard Hofmann, Neunkirchen a.B. (DE); Ulrich Bleitner, Ebersdorf bei Coburg (DE); Christian Blumberg, Erlangen (DE); Heribert Amtmann, Pretzfeld (DE)

(21) Appl. No.: 12/830,836

(22) Filed: Jul. 6, 2010

(51) Int. Cl. A61B 5/00

(52) U.S. Cl. 600/301

(57) ABSTRACT

A system and method of managing an integrated laboratory for diagnosing and treating a patient is described. The system is divided into a laboratory room and a cockpit control room. The cockpit control room has a plurality of workstations for display and control of the laboratory equipment, so that two operators may cooperate in operating the equipment without conflict. All of the needed data is displayed to each operator in a single display where the display area is divided by a predetermined selectable grid pattern. The equipment in the laboratory room and the cockpit control room connected by electrically isolated paths. A backup workstation is provided, where at least the essential laboratory equipment is connected using an independent electrically isolated path.

### TECHNICAL FIELD

The present application may relate to a system and method of integrating the monitoring and control of a variety of cooperating medical devices.

### BACKGROUND

In a modern medical facility, the people in the control room of a Cath-, EP-, or Angiolab is responsible for the operation and monitoring multiple modalities. Each of these modalities usually is associated with one or multiple separate displays and control devices for visualization of the data, which may include image data, and for control of the modality. This results in a substantial amount of required space in the control room to house the equipment providing for the interface and control of the various modalities, as well as for the display and control thereof. The amount of equipment depends on the number of installed modalities and the specific attributes of each modality.

A user of the different modalities will usually need to move physically from the display and control location of one modality to the other in order to control the desired modalities sequentially during the procedure. At the least, a plurality of displays will need to be present and consulted, as well as a plurality of joysticks, keyboards, pushbutton switches, computer mice, and the like. Some existing solutions to this problem display the different modality graphical user interfaces on a single large size flat panel display with a single keyboard and mouse control, but do not address the complex workflow and safety aspects of a control room.

Important aspects of the control room problem that are not solved by existing systems include: a) changing the focus of the control room from one modality to another as the user transitions from one modality to another; b) providing for integrated display of images and status from different modalities having different native display resolutions corresponding to the technology, convention and usage (e.g., 1280×1024 pixels, 1600×1200 pixels, 1920×1200 pixels); c) safety considerations for medical devices require electrically isolated connections between the examination room and the control room and within the control room, where modalities from different manufacturers or different design generations are co-located; d) during an examination or treatment there may be the need to control different modalities simultaneously; e) the control room may host the majority of modality controls and the modalities may need to be started or shut-down accordingly and automatically, if required; and, f) different modalities have diverse requirements regarding the type and or quality of the display: e.g., minimal resolution, medical grade quality, luminous intensity, and the like.

The focus of activity in the control room changes during the procedure from one modality to another. For example, a technologist in the control room usually documents the examination steps within the cardiovascular information system (CVIS). At a certain step in a procedure the physician may request the technologist to segment a region of interest in a 3D reconstruction dataset on a processing workstation. While it is of importance to focus on the CVIS most of the time, the technologist now has to work with the processing workstation and to enlarge the display of the workstation as much as possible allowing support for accurate and easy segmentation.

Different technicians or users need to have access to different modalities at the same time within the control room environment. There are usually different workplaces within a control room consisting of separate displays and controls to allow parallel work. That is, while one user finishes a procedure on the recording modality, the other user may have started with a reporting task.

### BRIEF SUMMARY

A system for managing the operation of a treatment laboratory is disclosed, including a plurality of medical devices, configured for imaging, treating, or monitoring a patient, wherein at least one device of the plurality of medical devices is an essential device. A control cockpit room has processing, control interfaces and display devices such as a primary workstation, configured to receive data from the plurality of medical devices, which may be in a treatment room, over a data connection and to display the data processed by a controller on a display.

A slave display may be configured to display the same image data as that displayed on the primary workstation, or to display other high resolution data. Control of the control and display system and the associated medical systems is effected through an input device. A backup workstation may be provided and would be electrically isolated from the primary workstation, and the essential equipment of the medical equipment may have a data connection to the backup workstation that may be independent of the data connection of the essential equipment to the primary workplace.

The data connections between medical equipment in the laboratory room and the control cockpit room provides for electrical isolation of the devices in the control cockpit room from devices in the laboratory room.

In an aspect, a method of operating a medical laboratory for diagnosing or treating a patient includes: providing a plurality of medical devices in a laboratory room, where at least one of the devices is an essential device; connecting the plurality of devices in the laboratory room to a cockpit control room, the connections providing for electrical isolation of the data paths between the laboratory room and the cockpit control room; providing a first workstation and a second workstation in the cockpit control room for displaying data from the medical device and controlling the medical equipment, only one workstation being enabled to control a specific medical device at the same time; providing a backup workstation having an independent data path to each essential device; operating the workstations such that an operator at each workstation views data of the medical device being actively controlled by the workstation, and the medical device being actively controlled by the other of the workstations, and indicating the data area of the medical device being actively controlled by the workstation.

A software program product is described, the product being stored on a computer readable medium, and including instructions for configuring a first workstation and a second workstation to display data from a plurality of medical devices on a single display at each workstation; coordinating the capabilities of the first and the second workstation so that only one of the first or second workstations is enabled to control a specific one of the medical devices at any time; partitioning the display of each workstation into a grid layout where image data or control interfaces for a selected one of the medical devices is displayed in a selectable area defined by the grid; accepting operator input through a control interface to select the specific medical device to be controlled, and to control the specific medical device using input from a keyboard and a mouse; and, retrieving data from an external data base of medical data, and displaying the retrieved data in an area of the grid that is selected by the operator.

### BRIEF DESCRIPTION OF THE DRAWINGS

FIG. 1 shows three examples of a display image area with different grid patterns defining areas in which data may be displayed;

FIG. 2A shows the left-hand grid pattern of FIG. 1 where specific image or control data has been assigned to the areas; and, FIG. 2B shows the resolution in pixels of the areas of FIG. 2A;

FIG. 3 shows the left hand grid pattern of FIG. 1, where the image in the upper left area is annotated to indicted that the data is displayed in the native resolution of the modality that is the source of the image data;

FIG. 4 shows an example of a display image area having a grid pattern of the left-hand example of FIG. 1, where image data and control function icons are displayed for the systems assigned to the specific display areas of the grid; and

FIG. 5 is a block diagram of a medical system having a treatment room and a cockpit control room.

### DESCRIPTION

Exemplary embodiments may be better understood with reference to the drawings. Like numbered elements in the same or different drawings perform equivalent functions.

In the interest of clarity, not all the routine features of the examples herein are described. It will of course be appreciated that in the development of any such actual implementation, numerous implementation-specific decisions must be made to achieve a developers' specific goals, such as consideration of system and business related constraints, and that these goals will vary from one implementation to another.

The combination of hardware and software to accomplish the tasks described herein may be termed a system. The instructions for implementing processes of the system and method may be provided on computer-readable storage media or memories, such as a cache, buffer, RAM, removable media, hard drive or other computer readable storage media. Computer readable storage media include various types of volatile and nonvolatile storage media. The functions, acts or tasks illustrated or described herein may be executed in response to one or more sets of instructions stored in or on computer readable storage media. The functions, acts or tasks may be independent of the particular type of instruction set, storage media, processor or processing strategy and may be performed by software, hardware, integrated circuits, firmware, micro code and the like, operating alone or in combination. Some aspects of the functions, acts, or tasks may be performed by dedicated hardware, or manually by an operator.

The instructions may be stored on a removable media device for reading by local or remote systems. In other embodiments, the instructions may be stored in a remote location for transfer through a computer network, a local or wide area network, by wireless techniques, or over telephone lines and be stored for local execution by the computer or system. In yet other embodiments, the instructions are stored within a given computer, system, or device.

Communications between the devices, the system, subsystems, and applications may be by the use of either wired or wireless connections. Such communications may include the use of a local area network (LAN), a wide area network (WAN) such as the Internet, the public switched telephone network (PTSN), or such other equivalent systems that exist or may subsequently be developed. Wireless communication may include, audio, radio, lightwave or other technique not requiring a physical connection between a transmitting device and a corresponding receiving device. While the communication may be described as being from a transmitter to a receiver, this does not exclude the reverse path, and a wireless communications device may include both transmitting and receiving functions. Wireless communication makes use of electronic hardware and antennas to radiate electromagnetic radiation which may be received by suitable antennas and electronic hardware and processed to obtain the information that has been transmitted.

A central control location that integrates status and operational displays of a number of interrelated systems, and the control thereof, may be termed a “cockpit”, by analogy to the integrated cockpits being developed for aircraft.

Some of the essential criteria and some of the desiderata for consideration in evolving a medical laboratory cockpit have been previously discussed; such criteria are by way of example so as to provide an exemplary embodiment of the concepts being claimed. From an analysis of the system specifications of each of the medical systems to be controlled by the cockpit, and the best practices for using each medical system, both individually and in concert, the display and control equipment may be configured from the cockpit hardware and cockpit system software to form one or more workstations.

Even when the basic design has been performed, the human factors and safety considerations taken into account, it is well known that individuals have preferences in how they interact with computer implemented systems. This may be accommodated by a limited number of standard configurations of the display and control apparatus, with the ability to simply switch between the configurations using the facilities of the display and human interfaces to the cockpit.

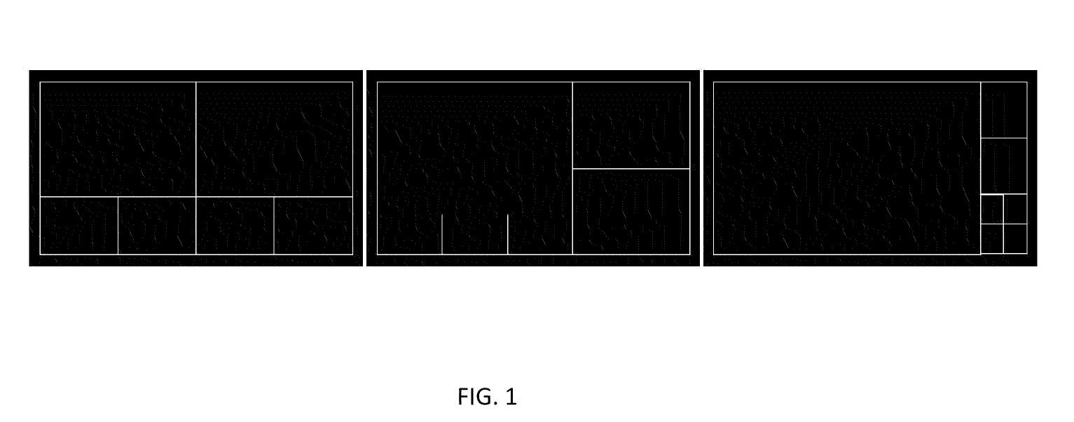

In this example, a large color display, suitable for displaying medical images at an appropriate resolution may be configured so as to partition the entire display into a plurality of smaller display areas that are compatible with each other. One may term the partitions as a “grid” having areas of predetermined size and location. In the present example, three different grid configurations are used, as shown in FIG. 1. One of the grid configurations may be selected as the default so that the cockpit system starts in a known stable state that is immediately familiar to all operators of the cockpit. The default grid configuration may be populated with images and control icons that are a standard initial operation configuration. This is helpful, as the state in which the cockpit was last configured is likely to be quite different from the state that may be needed when a patient is first brought to the laboratory for diagnosis and treatment. The startup display configuration and data assignments would usually be selected so that the remaining medical equipment may be statused and then configured for the particular medical protocol being performed. Such a configuration may then be altered by the operator as needed, within the system constraints.

Safety considerations are a significant aspect of the cockpit design, and essential devices or systems need to be monitored and controlled even if there is a failure of the primary display and controller. A backup display maybe provided for redundancy, and that display may be a monochrome display. Depending on the display technology, a monochrome display may also function to display images where the present generation of color displays may have either inadequate resolution, dynamic range, or the like.

As more than one technician may be involved in configuring, monitoring, and controlling the medical equipment, a second complete cockpit display and controller may be provided (a second workstation). Apart from the redundancy involved, the handover from one person to another may be made with both persons viewing the same display at the same time, albeit on two different monitors. Coordinated activities are facilitated as there is no discontinuity in attention of the personnel. Generally, different technicians are trained and skilled in the different sophisticated systems being used in conjunction with each other and either a handover of control, or coordinated efforts, may be desirable to support the activities of the physicians.

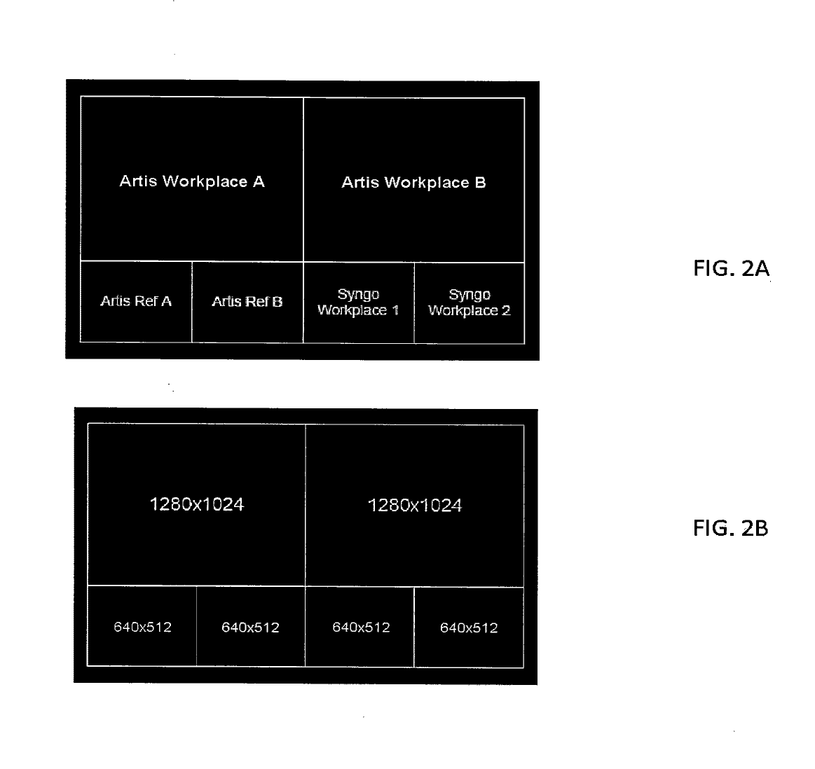

In an example, the left-hand grid display of FIG. 1 is shown in FIG. 2A where the various display areas have been assigned to specific data displays or control functions of two different medical systems. The resolution of the display areas assigned in FIG. 2A is shown in FIG. 2B.

However, where a system has a native (intrinsic) resolution requirement, such as 1600×2100 pixels, such a grid configuration may be also be provided when that system is part of the suite of equipment of the particular laboratory. In this grid configuration, for example, perhaps only the image display of the controlled system may be displayed, and the other systems represented by icons or low resolution displays. Providing such a variety of predetermined compatible grid configurations enables viewing images in the original resolution of each imaging modality when needed.

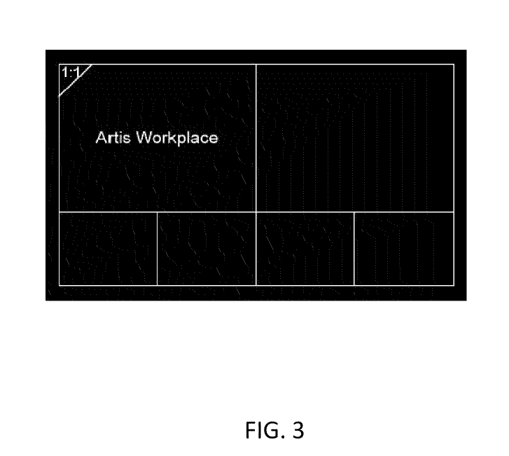

As not all of the images being displayed on the composite display of FIG. 3 may be in the native resolution, particularly where the particular imaging modality is in a standby state, not being directly controlled by the workstation, in a non-functioning state, or is not the focus of the display operators attention, providing an indication that the image is indeed in the native resolution may be useful. An indicator such as a “1:1” icon may be placed in an expected position of the display as shown in FIG. 3. By “expected position” a position that is consistent from display-area-to-display area is meant, so that the user does not have to check all areas of the displayed image to comprehend the present image resolution.

A second cockpit workstation position permits a second operator or technician to freely control one selectable modality of the available modalities so that a second modality may be operated at the same time as the first modality, and this may be desired when coordinating the operation of a catheter system with an imaging modality, for example. The two cockpit workstations positions are, however, configured such at only one cockpit workstation is capable of controlling a particular piece of equipment at a time. That is, the two operators are prevented from each controlling the same piece of equipment simultaneously. When an operator has selected a modality, the image or status associated with the modality is shown as being active on the operator display, and cannot be active on the second operator display unless the first operator has relinquished control of the device by a positive act. Thus the two operators may each control separate modalities, while monitoring the status and images of the modality controlled by the other operator.

A backup workstation may be connected directly to the different modalities using a different data path from that which connects the modalities to the other cockpit workstations. The data paths may be direct connections, wireless connections, a separate local area network, or the like. Such redundant connections are used to provide at least mandatory and patient safety information in the cockpit room in the event that the cockpit primary workstations become inoperative. This configuration ensures that each modality whose functioning may be essential to patient safety in the laboratory has at least this alternate means of monitoring and control.

In an example, Keyboard-Video-Mouse (KVM) switches and a high luminance monochrome display may be used at the backup workstation. By using the KVM switches the needed modalities may be selected and controlled sequentially and independently from control by the other cockpit workstations. Where the backup function is not in use, the display of the backup workstation may provide for the display of information with diagnostic image quality for angiographic images or other images where fine detail needs to be displayed.

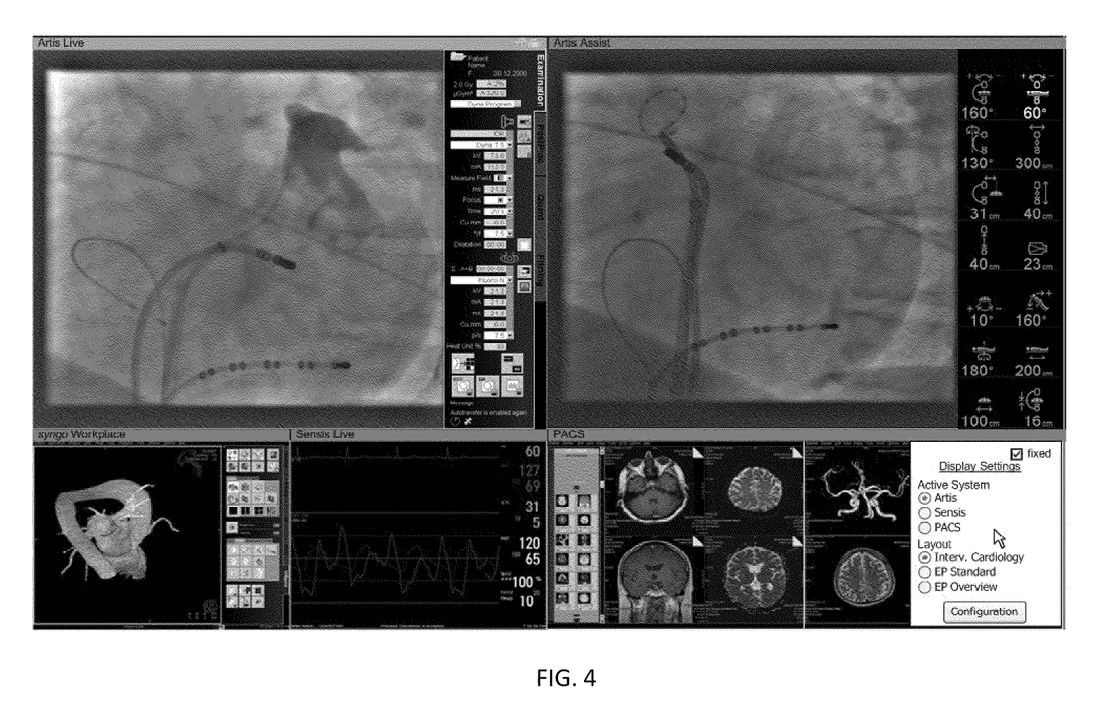

As shown in FIG. 4, an active display may display “live” fluoroscopic imagery in one axis (left-hand image pane) while the orthogonal axis image is shown in the right-hand image pane. Other data assisting the operator in visualizing the position of the patient with respect to the imaging modality is shown at the far right hand side of the composite display. In this instance, the two displays, being representative of the same area, are shown in the same scale and resolution so that they may be directly compared. Other information related to the image processing and display is shown associated with the left hand live display, and the keyboard and mouse may be used so as to adjust the values shown to optimize the display for the present task.

At the lower portion of the composite display, on the left-hand side, may be images previously obtained and stored in a PACS system, which is a DICOM (Digital Communications in Medicine) compatible data base management system. In this instance, computed tomographic (CT) images of the patient have been retrieved and displayed as slices, and segmented vasculature. Another segmented image, perhaps from a different imaging modality or image visualization process is shown at the left bottom side of the composite display. Between the two image groups at the bottom of the composite display, the patient vital signs are displayed, in this example. The collection of such a large amount of information regarding the patient in an easy to view form is important from a human factors viewpoint and improves the speed of decision making by the physician.

In this example, the far bottom right hand portion of the composite display is a control box that is in a “pop-up” state. That is, the box may ordinarily be minimized as an icon, however, if selected by the cursor, or a function key, or function switch, the box will exist in a pop-up state. In this example, the box is a control and configuration box enabling the operator to select a new display grid based on the operations to be performed, and to assert control of another modality. Radio buttons are used in each instance as only one of the modalities may be selected at a time. In an aspect, when a modality is being controlled by the other of the cockpit positions, the label of that modality may be grayed out so as to indicate that the device is not available for selection at the present time from the cockpit position.

The individual display areas may be interchanged with each other by, for example, the operator actuating an input device and using drag and drop techniques. The input device may be a mouse, joy stick, or track ball. Other input devices such as soft keys, dedicated keys of a keyboard, an alphanumeric keyboard, touch panel input devices, a touch sensitive display, or the like may be used. However, where the operator attempts to configure the display in manner that has been excluded by the system design, the display will immediately return to the previous acceptable state. Voice commands may also be used.

There may be default positions and display characteristics for various states of the system. For example, the modality that is being controlled by the workstation (the “active modality”) may be shown in the upper left hand portion of the composite display and have a different border. All of the other modality data is presented for context and reference, but the other modalities cannot be controlled unless the desired modality is made the active modality, by means previously described. Other techniques of asserting active control of a modality may be used. For example, positioning the cursor on an image area that is inactive and double clicking the mouse is a known method of indicating an operation to select the image as an active image. In this context, the activation of an image may also activate the control of the modality by the workstation while deactivating control of the previously active modality. Such a circumstance may occur, for example, when moving from a diagnostic phase to a treatment phase.

FIG. 4 also shows, associated with the lower-left-hand image, a set of icons that may be used to control the display of the modality data, or to control the operation of the modality when it has been activated. The keyboard icons may be shown in a larger scale for actual actuation by placing the mouse cursor in a designated corner or double clicking on the icon pad. Other means of activation of such a pad are known and may be used, including a function key and switches. While the control and selection of the devices and image data has been described as being by a mouse, touch screen technology is being developed and may be used in place of the mouse, or in addition to a mouse.

A repeater display for one or both of the cockpit consoles may be provided in the treatment portion of the laboratory so that medical personnel may view the same images as in the cockpit. The medical personnel may request that the cockpit operator retrieve, display, and manipulate images from the modalities, whether the images are real-time images, or archive images of the patient, during the course of the procedure.

Modern C-arm X-ray systems such as the ARTIS zee (available from Siemens AG, Munich, Germany) may be equipped with flat-panel detector (FD) technology, and the C-arm may be mounted to a ceiling or a robot for enhanced accessibility and maneuverability. The data obtained may be used as fluoroscopic data in real time, or processed to yield computed-tomography-like (CT) images.

In an aspect, the ARTIS zee system may be configured as a bi-plane X-ray system to obtain fluoroscopic images in orthogonal planes so as to aid in the visualization of interventional apparatus such as catheters with respect to body structures.

In another aspect, the ARTIS zee may be integrated with the Stereotaxis, Inc. (St. Louis, Mo.) NIOBE Magnetic Navigation System, so as to provide magnetic sensing for the guidance of catheter- and guidewire-based devices along complex paths within the heart and coronary vasculature. Other guidance systems may also be used.

Other interventional or diagnostic equipment including, but not limited to, ablation catheters, power injectors, or acoustic imaging may be employed. 3D ultrasound data (such as echocardiography data), intra-cardiac echocardiography (ICE), extra-corporal data (such as trans-thoracic echocardiogram (TTE), or trans-esophageal echocardiogram (TEE) data may also be used, and the relevant equipment may be located, in part, in the laboratory room, and controlled or monitored from the cockpit room. The patient vital signs may be monitored by a system such as the Siemens Sensis system. The specific equipment that is used in the laboratory may vary depending on the overall capability and flexibility desired in the design and use of the laboratory.

Previously obtained CT (computed tomography), MRI (Magnetic Resonance Imaging), X-ray data, electrophysiology data, and the like, may be used in conjunction with the fluoroscopic images and the other laboratory sensors to perform the diagnosis and to guide the interventional apparatus to treat the patient. Previously obtained image data of the patient may be retrieved, for example, from a Siemens PACS (Picture Archiving and Communications System) station, which may be a DICOM (Digital Imaging and Communications in Medicine) compatible data base system. Such data may be retrieved either from a local data base or a remotely located data base using networking technology, such as a local area network (LAN), the Internet, or the like.

Where this variety of equipment and data sources has been accessed or displayed on monitors and using interfaces that are particularized to the individual systems in existing systems, the lack of an integrated display and control console is inefficient and may lead to errors. When the system control and monitoring is merged into a composite display where the attributes of each system with respect to display and control are respected, a small number of technicians may effectively support the medical personnel in the use of the systems in diagnosis and treatment. The collation of data and reports for storage and distribution may also be facilitated.

By connecting each of the monitored and controlled systems to the cockpit workstations through electrically isolating circuitry, such as optical isolators, for example, the individual systems, which may have differing electrical specifications, grounding and other safety-related requirements, may be operated together while maintaining the appropriate physical and electrical isolation. Such electrical isolation simplifies the integration of the laboratory.

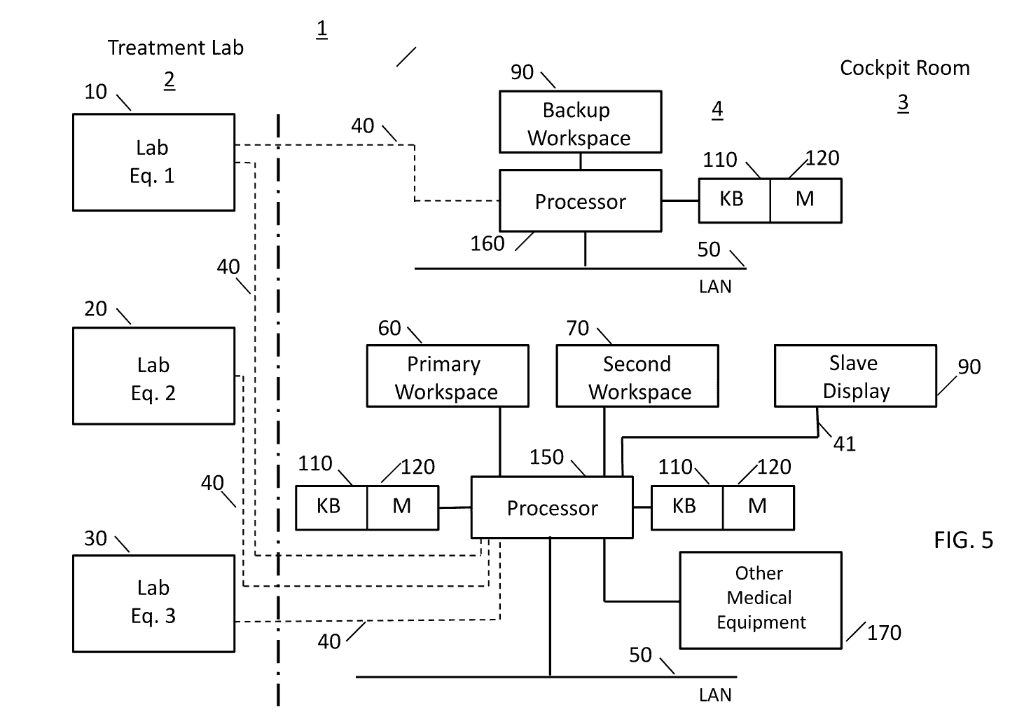

FIG. 5 is a block diagram of medical treatment facility using the cockpit principle. Only a representative group of equipment is shown, as the equipment that could be connected together and operated from the cockpit may be selected from a large number of individual equipment types so as to optimize the design of a particular laboratory.

In an aspect, the integration of the display and control of the laboratory in a cockpit may permit the introduction of newly approved equipment, or the upgrade of an equipment type with a small effort, as the display format and protocol would have already been established. This would also contribute to lowered training costs.

The system may be divided into the treatment laboratory 2 and the cockpit 3. The treatment laboratory 2 is where the patient is diagnosed and treated, and has the sensing and treatment portions of the laboratory equipment, such as the C-arm X-ray system source and flat-panel detector, catheter system, patient support table and the like, as is known in hospital systems. Local control of some of the equipment is possible, depending on the design of the facility, and the sensed data may be processed locally to the equipment in the laboratory, or the data may be processed in the cockpit room, or elsewhere. Each equipment type may have a different location for the sensing and the processing, so the details thereof are not shown.

The laboratory equipment may be broadly categorized as being essential equipment 10, diagnostic equipment 20, and treatment equipment 30, although there may be equipment that is used for both diagnosis and treatment. Other medical equipment 170 may be located in the cockpit room or elsewhere outside of the treatment laboratory. The laboratory equipment 10, 20, 30 may be connected to the control room 2 using a variety of data communications methods. For ease of illustration, such data and control paths are shown as a line 40 connecting the equipment and the control room. The characteristics of the line 40 may be a universal serial bus (USB), Ethernet, or other interface as is known or may be subsequently be developed for the purpose. Each of the lines should be of a type that provides electrical isolation consistent with the safety and other design criteria. Each of the individual equipments in the laboratory 2 is connected to an interface of a USB/Video processor 150 in the cockpit room 3. In this manner the individual equipments are isolated from each other.

In the case of essential equipment 10, a second line 40 is provided so as to connect to a separate backup workstation 4 in the cockpit room 3. The backup workstation 4 may be a subset of the functionality of the primary and second workstations 60, 70. However, the data from the essential equipment 10 and the processing by a processor 160, and the control using a keyboard 110 and a mouse 120 or other input devices, is physically separate from that of the remainder of the cockpit. This independence may extend to having a separate source of prime power, and uninterruptable power supply, or the like, so as to provide essential functionality in an equipment-related emergency.

The laboratory equipment 10, 20, 30 may communicate with the processor 150 of the cockpit workstation 60, 70. The processor 150 may be a single processor, or a plurality of processors with differing functions and capabilities and a variety of interfaces so as to be compatible with the controlled equipment, and a the processor may have either integral data storage, or may be capable of accessing data and program storage, which may be local or remotely located. The processor 150 may be, for example, include a universal serial bus (USB) switch which may also have video switching capabilities, or the functions may be allocated to a number of hardware and software elements which, together comprise the processor 150. The processor 150 may route the signals appropriately, and provide for rapid reconfiguration when needed. Each of the primary 60 and second displays 70 may be a color monitor having the size and resolution appropriate for the design. The video interfaces 41 may of a conventional type, at the time of system design, such as HDMI, or other types as are now known, or may later be developed, so as to permit upgrading of the monitors without further equipment modifications. The processor may have an interface to a local area network (LAN) 50 so as to either interface with other equipment or to communicate with other systems either in the cockpit of remotely located therefrom. Such communications may include the use of the Internet or other wide area network (WAN).

One or more slave displays 80 may be provided, and these displays may be of a high-performance black and white design so that they may also be used for viewing high-resolution high-dynamic-range images obtained by the imaging modalities. Such a slave display 80 may also be provided in the treatment lab 2 to assist the medical personnel in controlling or directing the control of the laboratory equipment, and to view ancillary data, such as may be retrieved from a DICOM data base. When the slave display 80 is located in the treatment room 2, the connection 41 may be provided with electrical isolation. Often this isolation is provided by using optical data transmission paths. The optical data transmission paths may typically be over optical fibers, with transceivers at each end of the path.

The system 1 is provided with software instructions for execution by the processors 150, 160 and by the configuration of the individual equipments so as to perform functions previously described herein.

Although only a few examples of this invention have been described in detail above, those skilled in the art will readily appreciate that many modifications are possible without materially departing from the novel teachings and advantages of the invention. Accordingly, all such modifications are intended to be included within the scope of this invention as defined in the following claims.

What is claimed is:

1. A treatment laboratory, comprising:
a plurality of medical devices located in a laboratory room, configured for at least one of imaging, treating, or monitoring a patient; and
a control cockpit room, having processing, control interfaces and display devices comprising:
a primary workstation having a display, configured to receive data from the plurality of medical devices over a data connection, to display data processed by a workstation processor, and to receive control inputs from an operator interface;
wherein data connections between medical equipment in the laboratory room and the workstations provide for an electrical isolation of the equipment in the control cockpit room from that in the laboratory room.

2. The system of claim 1, further comprising:
a backup workstation, electrically isolated from the primary workstation, wherein one of the medical devices is an essential device, and the essential device has a data connection to the backup workstation that is independent of the data connection of the essential device to the primary workstation.

3. The system of claim 2, wherein the electrical isolation is provided by an optical fiber.

4. The system of claim 1, wherein a second workstation is provided with the same capabilities as the primary workstation.

5. The system of claim 4, wherein the primary workstation and the second workstation are each capable of controlling a same group of medical devices, wherein only one of the primary workstation or the second workstation is enabled to control a specific medical device of the plurality of medical devices at a specific time.

6. The system of claim 5, wherein image data or control data of the specific medical device being controlled by each workstation is designated by a highlighted aspect of the display of the controlling workstation.

7. The system of claim 5, wherein a display format of the display of the workstation is configurable so as to provide a plurality of predefined data areas for display of data from the medical equipment or data sources configured to be monitored or controlled from the workstation.

8. The system of claim 6, wherein the display format comprises a grid having at least one area with a display resolution compatible with a native data display resolution of a source of image data being displayed.

9. The system of claim 7, wherein each of the grid areas is assignable to a selected one of the medical devices, and the assignment is changeable using a drag and drop function of a computer mouse-type interface.

10. The system of claim 7, wherein a plurality of grid configurations are predetermined, and one of the plurality of grid configurations is selectable by an operator input.

11. The system of claim 1, wherein one of the plurality of medical devices is a C-arm X-ray device.

12. The system of claim 1, wherein the essential device is a vital signs monitor.

13. The system of claim 1, wherein the control inputs are provided by an input device.

14. The system of claim 13, wherein the control inputs are provided by programmed key switches.

15. The system of claim 1, further comprising:
a slave display configurable to display the same image data as that displayed on the primary workstation display.

16. A method of operating a medical laboratory for diagnosing or treating a patient, the method comprising:
providing a plurality of medical devices in a laboratory room, wherein at least one of the devices is an essential device;
connecting the plurality of devices in the laboratory room to a cockpit control room, the connections providing for electrical isolation of the data paths between the laboratory room and the cockpit control room;
providing a first workstation and a second workstation in the cockpit control room for displaying data from the medical device and controlling the medical equipment, only one workstation being enabled to control a specific medical device at the same time;
providing a backup workstation having an independent data path to each essential device;
operating the workstations such that an operator at each of the first and the second workstation views data of the medical device being actively controlled by the workstation, and the medical device being actively controlled by the other of the first and the second workstations, and the data area of the medical device being actively controlled by each of first and the second workstation is indicated.

17. The method of claim 16, wherein one of the medical devices is a C-arm X-ray device, and an essential device is a vital signs monitor.

18. A software program product, stored on a computer readable medium, comprising:
instructions for configuring a first workstation and a second workstation to display data from a plurality of medical devices on a display at each workstation;
coordinating the capabilities of the first workstation and the second workstation so that only one of the first workstation or second workstations is enabled to control a specific one of the medical devices at any time;
partitioning the display area of each workstation into a grid layout where image data or control interfaces for a selected one of the medical devices is displayed in a selectable area defined by the grid;
accepting operator input through a input device to select the specific medical device to be controlled, and to control the specific medical device using input data from the input device.
